(19) United States
(12) Patent Application Publication
(10) Pub. No.: US 2021/0198622 A1
(43) Pub. Date: Jul. 1, 2021

(54) CONTROLLED AGGREGATION OF CELLS

(71) Applicants: Jozef Stefan Institute, Ljubljana (SI); Institut za metagenomiko in mikrobne tehnologije, d.o.o., Ljubljana (SI)

(72) Inventors: Ales LAPANJE, Ljubljana (SI); Tomaz RIJAVEC, Ljubljana (SI)

(73) Assignees: Jozef Stefan Institute, Ljubljana (SI); Institut za metagenomiko in mikrobne tehnologije, d.o.o., Ljubljana (SI)

(21) Appl. No.: 17/137,557

(22) Filed: Dec. 30, 2020

(30) Foreign Application Priority Data
Dec. 31, 2019 (GB) 1919467.9

(51) Int. Cl. C12N 5/00

(52) U.S. Cl. CPC C12N 5/0018; C12N 2533/30; C12N 2533/78; C12N 2533/74

(57) ABSTRACT

A method for topographically controlled aggregation, wherein cells of the same type or different type, such as different strains of bacterial cells, are layered in a way that controls the size, structure and number of aggregates as well as the potential spatial distribution of the different species of cells within the aggregates. The method includes a step of surface modifying cells with a polyelectrolyte.

### FIELD

The aspects of the disclosed embodiments relate to cell aggregation and particularly, although not exclusively, to microbial or bacterial cell aggregation.

### BACKGROUND

Recently, microbiological research has indicated that the individual placement of particular cells is important for community functioning, since the microbes can transfer metabolites more efficiently, produce biofilms and develop particular niches based on the local physicochemical properties.

These insights are not important only from the academic perspective, but are also extremely important for biotechnological applications in different biotechnology fields such as food biotechnology, remediation, biofouling etc. For example, the topographical placement of individual cells in crude cheese influence the taste, structure, ripening as well as ageing of the cheese.

Aggregates are important in the biofertilisation of crops by allowing different strains to be used in a very small space. Aggregates formed from strains that can fix nitrogen, dissolve inorganic material to release nutrients, or produce protecting substances and prevent diseases, can improve the growth of the plant and increase production of plant biomass.

Aggregates are also important in the purification of waste water or tap water contaminated with pesticides, especially when purification involves aerobic and anaerobic steps. The surface of the aggregates enables aerobic activity of microorganisms, whereas the internal part of the aggregate promotes anaerobic respiration and fermentation. Use of aggregates enables better colonisation of the sand particles due to the easier retention of aggregates within the sand particles. Retention of the aggregates is increased as a result of the larger size of aggregates compared to the individual cells (mm sized aggregates compared to μm sized cells).

Recently, multi stage biotechnological fermentation have been replaced by one fermentation processes using co-cultures. The co-culture is hard to control when cells are in the suspended form due to different growth kinetics. Moreover, currently the co-cultures are mainly used in a process when it is necessary:
1. To induce production of secondary substances, here the induction relies on interspecies interaction or quorum sensing compounds acting in trans.
2. To scavenge oxygen for fermentation of carbon hydrates to short fatty acids such as acetate and lactate.
3. To involve complex, but cheap substrates (e.g. molasses, cellulose, lignocellulose), which can be used for the production of biofuels, acetate, lactate.
4. To produce complex secondary metabolites involving large metabolic pathways, for which genes cannot be easily cloned into the biotechnologically applicable host cells,
5. To directly transfer autotrophically produced carbohydrates to the heterotrophic cells.

These systems of co-culture are hard to optimise due to different growth dynamics of the strains and increased distances between cells when complex substrates are broken down to the level that is acceptable for use by producers of substances. Scavenging oxygen is hard to monitor within the whole culture and currently there are no easy ways to do this. Also the co-culture process is often separated into two steps, aerobic and microaerophylic/anaerobic. The direct combining of an autotrophic process with a heterotrophic process is currently only possible in suspensions or formed capsules of genetically modified autotrophic organisms which are capable of transmembrane exporting of carbohydrates. It is limited in efficiencies due to the large distances between the cells.

In contrast, aggregation enables close contact between cells and more intensive interactions. The interactions facilitate (i) exchanging growth substrates, secondary metabolites as well as quorum sensing molecules, (ii) scavenging oxygen due to the local increase of the oxygen consumption, enabling formation of anaerobic niches within the aggregates (e.g. natural cosms in the biologically water treatment processes).

Important factors in cell aggregation are the size of the aggregates, distribution of cells within the heterocellular aggregates (aggregate structure) and number of the aggregates. Size of the aggregate determines the consumption of nutrients and flux of gases within the layers of the aggregates, and also determines the distribution of the aggregated cells in the fluid bed, slow/quick sand filters, or other sorts of column type bioreactors. Size of the aggregate also determines the sedimentation of the biomass. Easier sedimentation also simplifies multiple reuse of the biomass in the fermenters.

The structure of the aggregates determines how cells are distributed within the aggregate. This is extremely important since the distribution of different types of cells determines the cascades of the biotechnological processes. Cells closer to the surface are involved in the earlier steps (e.g. degrading lignocellulose) than cells deeper within the aggregates which are involved in the later steps (e.g. fermentation and production of biofuels or acetate, lactates). Moreover, oxygen scavenging occurs in the most outer layers of the aggregates, enabling anaerobic processes to occur within the aggregates (e.g. nitrification and denitrification coupling of different microbial cells).

U.S. Pat. No. 6,699,501 describes a process for encasing a biological or amphiphilic template in a capsule composed of consecutive layers of oppositely charged polyelectrolytes. The template can be disintegrated to leave the case intact. 2-40 layers can be deposited.

U.S. Pat. No. 6,967,085 describes using polyelectrolytes of two different viscosities to flocculate cell material. These can be added simultaneously, sequentially, or as a pre-formed blend. One polyelectrolyte is cationic and the second polyelectrolyte is cationic or substantially non-ionic.

U.S. Pat. No. 9,371,554 describes a method for harvesting recombinant protein by flocculating cells using cationic polyelectrolytes, allowing to settle, then removing supernatant.

IN 217966 B describes a process for artificially flocculating yeast particles by addition of cationic polymer material and a divalent metal ion.

Fakhrullin R F, Zamaleeva A I, Minullina R T, Konnova S A, Paunov V N., “Cyborg cells: functionalisation of living cells with polymers and nanomaterials”, Chem Soc Rev., 2012, Jun. 7; 41(11):4189-206 describes living cells with polyelectrolyte coatings, magnetic and metal nanoparticles or hard mineral shells. It describes methods for functionalisation of cells with polymers and nanoparticles and talks about cell viability and cell toxicity.

Fakhrullin R F, Lvov Y M., ““Face-lifting” and “make-up” for microorganisms: layer-by-layer polyelectrolyte nanocoating”, ACS Nano., 2012, Jun. 26; 6(6):4557-64 describes sequential adsorption of oppositely charged polyelectrolytes onto living biological cells in mild aqueous conditions.

Wang S K, Wang F, Hu Y R, Stiles A R, Guo C, Liu C Z., “Magnetic flocculant for high efficiency harvesting of microalgal cells”, ACS Appl Mater Interfaces., 2014, Jan. 8; 6(1):109-15 describes synthesis of a magnetic flocculent using iron oxide and cationic polyacrylamide. This was added to algal broth and used to harvest microalgal cells post-flocculation, with a magnet.

The aspects of the disclosed embodiments have been devised in light of the above considerations.

### SUMMARY

In contrast to the previous work, the present inventors have devised a method for topographically controlled aggregation, wherein cells of the same type or different type, such as different strains of bacterial cells, are layered in a way that controls the size, structure and number of aggregates as well as the potential spatial distribution of the different species of cells within the aggregates.

Current status of the knowledge in the field shows two perspectives (i) that separate bacterial cells can be modified using polyelectrolytes of high charge densities and (ii) the suspension of bacterial cells can be aggregated spontaneously and randomly.

Discussed herein are the surface potentials of cells. In general, cells naturally have a negative surface potential. Through surface modification, as explained herein, this can be switched to a positive surface potential, or enhanced to become a more negative surface potential. Cells with a negative surface potential are sometimes referred to herein as “negatively charged cells” for convenience. Similarly, cells with a positive surface potential are sometimes referred to herein as “positively charged cells” for convenience. This discussion of positive or negative refers to the effective surface potential of the cell, rather than an internal or intrinsic property of the cell itself.

In a first aspect, the disclosed embodiments provide a method for making an aggregation of cells, the method comprising the steps of (a) treating a first portion of cells with a positively charged polyelectrolyte to form a first portion of surface modified cells and (b) mixing the first portion of surface modified cells with a second portion of cells, to form aggregations of cells each having a negative surface potential and each comprising a layer of cells on the surface of a surface modified cell.

In some embodiments the second portion of cells is not surface modified.

In some embodiments the second portion of cells comprises a second portion of surface modified cells formed by a method comprising the steps of (i) treating cells with a positively charged polyelectrolyte and then treating said cells with a negatively charged polyelectrolyte, to form the second portion of surface modified cells. These steps can modify the natural negative charge of the surface of the cell in order to promote the formation of aggregates.

In some embodiments the first portion of surface modified cells or the second portion of cells is treated with magnetic particles to coat their surface with such particles or embed such particles onto their surface, before the step of mixing the first portion of surface modified cells with the second portion of cells. This permits the aggregates to be separated easily using a magnet.

In some embodiments the magnetic particles are paramagnetic.

In some embodiments the magnetic particles are Fe₃O₄ particles.

In some embodiments each positively charged polyelectrolyte is individually selected from polyethyleneimine, polydiallyldimethylammonium chloride, poly(allylamine hydrochloride), chitosan, cationic starch and amino cellulose.

In some embodiments each negatively charged polyelectrolyte is individually selected from polystyrene sulfonate, polyacrylic acid, alginate or cellulose.

In some embodiments the first portion of cells and the second portion of cells are of different types. Using different types of cells enables the use of different cellular processes in the same small space.

In some embodiments each cell is a microbial cell, preferably a bacterial cell.

Another aspect of the disclosed embodiments is a method for making an aggregation of cells, the method comprising the step of treating an aggregation of cells with a negative surface potential, as made by the method of any preceding claim, with a portion of cells having a positive surface potential, to form an aggregation of cells each having a positive surface potential and each comprising a layer of cells having a positive surface potential, on the surface of a layer of the second portion of cells, on the surface of a surface modified cell. By this method a multi-layered cellular aggregate can be formed.

The aspects of the disclosed embodiments also relate to cell aggregates made by the methods described herein.

The present disclosure includes the combination of the aspects and preferred features described except where such a combination is clearly impermissible or expressly avoided.

### SUMMARY OF THE FIGURES

Embodiments and experiments illustrating the principles of the present disclosure will now be discussed with reference to the accompanying figures in which:

FIG. 1 shows the preparation of artificially constructed multicellular aggregates produced from two different types of bacterial cells. (1) The surface charge/potential of the first cell type is altered to be positive using a layer by layer approach. The altered surface charge of the cell enables its attachment to the negatively charged cells of the second cell type. (2) The appropriate procedure of mixing and ratio of positively to negatively charged cells results in formation of aggregates consisting of different combinations and number of cells.

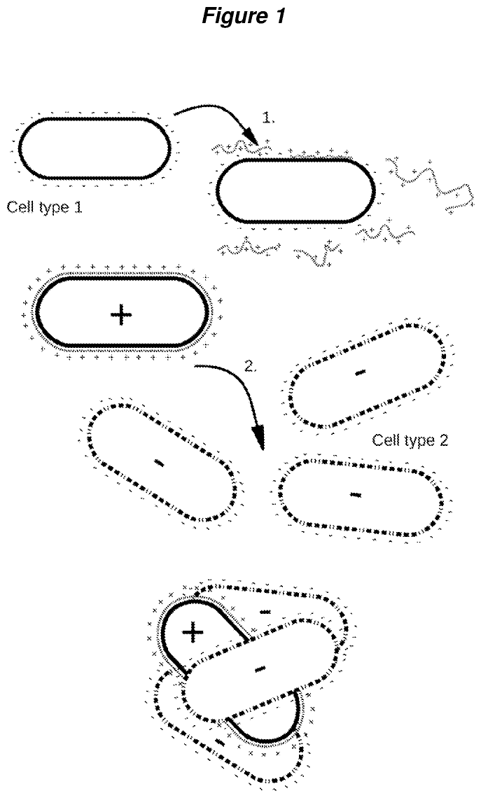

### DETAILED DESCRIPTION

Aspects and embodiments of the present disclosure will now be discussed with reference to the accompanying figures. Further aspects and embodiments will be apparent to those skilled in the art. All documents mentioned in this text are incorporated herein by reference.

In the present disclosure, a first set or portion of cells is made to have a positive charge (that is, a positive surface potential). Those cells are then mixed with a second set or portion of cells, which have a negative charge (that is, a negative surface potential). When positive cells are added slowly to negative cells, the different charges/potentials mean that the negative cells agglomerate around the positive cell. If negative cells are added slowly to positive cells, the positive cells agglomerate around the negative cells.

The present disclosure describes techniques for controlling the surface potential/charge (both type, positive or negative, and magnitude) of cells, by use of suitably charged polyelectrolyte coatings for surface modification. Thus aggregates comprising the desired cells in the desired layering are possible.

Furthermore, the present disclosure describes techniques by which the cells can be multiply surface modified by addition of several polyelectrolytes. This enables the layers of the eventual aggregate (which will comprise those cells) to be engineered as desired.

Furthermore, the present disclosure describes techniques by which the aggregates themselves can be made to the desired size and layering.

The present disclosure also describes techniques for making the aggregates magnetic, thereby improving the separability and ultimately the yield of them.

It is noted that in all the procedures described herein, the temperature of the cells is suitably kept within the range of 0 to 37° C.

Cell Types

Cells, for example microbial or bacterial cells, are well known. The present disclosure can be used with any types of cells, although it is particularly applicable to bacterial cells.

Cells generally have a naturally negative surface potential (naturally negatively charged). Suitably, the cells naturally have a surface potential of −100 to −10 mV, for example around −30 mV.

Examples of suitable cell types include bacteria cells. Any bacteria type can be used, provided conditions for deposition of polyelectrolyte layers are determined by establishing the suitable stage of growth, surface pre-treatment, suitable centrifugation speed, and toxicity of different polyelectrolytes. Examples of suitable bacteria cells are Lactococcus lactis, Pseudomonas spp., Escherichia coli, and Bacillus spp., Nitrobacter winogradskyi.

In the present disclosure, a first set or portion of cells are made to have a positive charge (that is, a positive surface potential). Those cells are then mixed with a second set or portion of cells, of the same or different type, which have a negative charge (that is, a negative surface potential). Depending on the mixing conditions, the different charges/potentials mean that the negative cells agglomerate around the positive cells or the positive cells agglomerate around the negative cells.

The cells used in the first set or portion of cells and the second set of cells may be the same or different. For example, the sets or portions of cells may be of the same bacterial strain, or of different bacterial strains.

Thus, the present disclosure allows aggregation of a layer of bacterial strain A on/around bacterial strain B by suitable surface modification and mixing. Similarly, a layer of a further bacterial strain (the same or different) can be then layered on that, giving a multiple-layered structure with each layer comprising cells which may be the same as or different from the cells in any other layer of the aggregate.

Cell Selection

Ideally, although not essentially, the cells used in the present disclosure can be selected such that they will have properties best suited to the aggregation procedure.

For example, by selecting cells which are at an appropriate growth stage, the cells may have low electrophoretic softness (λ⁻¹) as well as a high surface charge (ZN).

Cells can be cultivated, taken from different growth stages (for example, late lag, mid exponential and early stationary) and measured for electrostatic mobility dependent on the ionic strength using solutions of monovalent ions (for example, NaCl solution from 0.02 to 0.12 M by 11 measurements with 10 fold increased concentrations of ions).

The obtained curves can then be used as a basis for the calculation of the λ⁻¹ and ZN using the Ohshima model.

Before the measurement, the cells may be washed by centrifugation. There may be 1-5 such washings, for example 3 washings. The centrifuge may be, for example, at 3000 g for 3 mins). The cells may then be resuspended, for example in a 0.00062M NaCl solution prepared in deionised water (18 MOhm), and dispersed using, for example, maximum vortexing of the suspension of cells for 10 s.

When the appropriate growth stage is determined (that is, by considering the λ⁻¹ and ZN at different growth stages and assessing which gives the ideal balance of λ⁻¹ and ZN), the bacterial cells for use in the present disclosure can be prepared from a fresh culture.

A suitable protocol is that the overnight liquid cultures obtained from a single colony grown on solid medium are taken and 1 mL of this culture is transferred into a 100 mL fresh medium and incubated until reaching the optical density/ies corresponding to the previously determined appropriate growth stage.

A suitable such optical density may be, for example, an OD660 nm of 1.0 to 1.5, for example 1.1 to 1.3, and suitably around 1.2.

### Surface Modification—Switching the Surface Potential of Cells

In order to create cells with a positive charge (positive surface potential), the cell surfaces must be modified. In the present disclosure this is done by using a polyelectrolyte.

In order to change the negative surface potential of a cell to a positive one, a positively charged polyelectrolyte is used to treat the cells. It is attracted to the surface of the cell and forms a positively charged coating or layer; this renders the cell itself as having a positive surface potential (positively charged).

It will be appreciated that the same technique can be used to change the surface potential of a cell which has been surface modified to have a negative surface potential, as explained in more detail below.

For example, a suitable surface potential for such coated cells might be 10-100 mV, for example around 30 mV.

Various positively charged polyelectrolytes are suitable for use here.

Depending on the target surface potential/charge, the positive polyelectrolyte can be selected suitably.

For example, the positive polyelectrolyte may be one with a high charge density of, for example, 1 chargeable moiety per 3 heavy atoms (for example polyethyleneimine), or one with a moderate charge density of, for example, 1 chargeable moiety per 10 heavy atoms (for example, chitosan), or one with a low charge density of, for example, 2 to 5 chargeable moieties per 100 heavy atoms (for example, cationic starch). As used herein, a heavy atom is one that is not hydrogen. Other examples of suitable positive polyelectrolytes are polydiallyldimethylammonium chloride and poly(allylamine hydrochloride).

A positively chargeable moiety is one which may be protonated (such as N, NH or NH₂ in tertiary, secondary and primary amines respectively). A negatively chargeable moiety is one which may be deprotonated (such as SO₃H or COOH). For example, a suitable positively chargeable moiety may have a pKa of 6.5 to 11 and a suitable negatively chargeable moiety has a pKa of 1 to 7.5.

The cells are coated with a layer of the positive electrolyte by, for example, mixing solutions thereof. Before mixing with the positive polyelectrolyte solution, the cells may be subjected to treatment to break up any existing flocculation. Such treatment may be, for example, by ultrasound. Suitable conditions for that are 80 kHz, 100% power, for 3 minutes.

A positive polyelectrolyte solution of 0.025 to 0.25% v/w at pH 7 (adjusted by using, for example, acid such as HCl) may suitably be added to a solution of the cells to be modified. That is, the amount of positive polyelectrolyte in the positive polyelectrolyte solution may be 0.025 to 0.25% w/v, suitably 0.06% to 0.25% w/v.

The positive polyelectrolyte solution may be added to the cell solution at around 1:1 v/v.

The mixed solution can be left to stand for the coating to occur, for example at room temperature for 5 minutes.

After coating, any excess positive polyelectrolyte can be removed. This may be done by, for example, centrifugation (for example at 700 to 1300 g, chosen appropriately depending on the cells and polyelectrolyte present; the centrifugation step may be, for example, around 2-3 minutes). The obtained pellet is washed by removing the supernatant and slowly adding a new solution of appropriate ionic strength and pH to fill the whole centrifuge tube three times, being careful not to disturb the pellet. It will be appreciated by those skilled in the art that an appropriate ionic strength and pH may be selected to enable solubility of the polyelectrolyte and high survivability of the cells. This will vary depending on the types of cell and polyelectrolyte.

An appropriate positive polyelectrolyte and its concentration for mixing can be determined by the minimal inhibitory method using different amounts of polyelectrolytes and a standard amount of cells (around 100 CFU in 200 μl of media). By this method, cells are firstly washed and the number of cells is determined via optical density. This suspension of cells is added to the polyelectrolyte solutions in 1:1 ratio (v/v). The suspension is washed via centrifugation method to remove all the polyelectrolytes from the solutions and then the zeta potential is measured using electrophoretic light scattering (ELS) method. Polyelectrolyte solution is diluted two fold from 0.5 to 0.003. At each dilution the cells are added to the polyelectrolyte solution and the washing procedure repeated and the zeta potential measured. The decreasing power of surface charge is correlated with the amount of charged polyelectrolytes attached on the bacterial cells. Relevant concentrations are based on the determined electromobilities per cell:polyelectrolyte concentrations, which are suitably above 5 mV.

The concentration window of polyelectrolytes and charge densities is accordingly determined within the limits of the concentrations allowing the cells to survive while still being sufficient to change the surface potential to be positive.

### Surface Modification—Enhancing the Charge of Cells

While cells generally have a naturally negative charge (surface potential), it may be advantageous to increase it, that is, make it more negative. For example, this might enhance the strength of interaction with the positively charged cells, leading to better or stronger aggregation.

Where this is desired, positively charged cells (for example, made as explained above) can be treated with a negatively charged polyelectrolyte in order to apply a layer of negatively charged polyelectrolyte to them. This negative polyelectrolyte can impart an effective negative charge (negative surface potential) on the cell which is ‘greater’ (more negative) than that which it naturally had.

Suitable negative polyelectrolytes include, for example, polystyrene sulfonate, polyacrylic acid, aliginic acid and cellulose.

As with the positive polyelectrolyte, the negative polyelectrolyte can be layered onto the positive cells by mixing a solution of it with a solution of positively charged cells (for example as made above).

The pH of the negative polyelectrolyte solution may be adjusted, for example to about 7, using an alkali such as NaOH.

A negative polyelectrolyte solution of 0.025 to 0.25% w/v at pH 7 may suitably be added to a solution of the cells to be modified. That is, the amount of negative polyelectrolyte in the negative polyelectrolyte solution may be 0.025 to 0.25% w/v.

The negative polyelectrolyte solution may be added to the (positive) cell solution at around 1:1 v/v.

The mixed solution can be left to stand for the coating to occur, for example at room temperature for 5 minutes.

After coating, any excess negative polyelectrolyte can be removed. This may be done by, for example, centrifugation (for example at 1300 to 2200 g, chosen appropriately depending on the cells and polyelectrolyte present; the centrifugation may be for, for example, around 2-3 minutes). A higher gravitational force is used than for the centrifugation of the positive cells mentioned above.

The obtained pellet may then be washed to further remove polyelectrolyte by removing the supernatant and slowly adding a new solution of appropriate ionic strength and pH three times, being careful not to disturb the pellet.

Forming Aggregates

In the present disclosure, aggregates are generally formed by mixing positively charged (positive surface potential) cells with negatively charged (negative surface potential) cells. These may be made by the methods explained above. The negatively charged (negative surface potential) cells may be natural cells, that is, cells with no alteration of their surface potential. Alternatively, they may be cells which have a positively charged polyelectrolyte surface modification and an external negative polyelectrolyte surface modification.

The mixing of the positively charged cells and the negatively charged cells may be done in any manner. For example, a solution of the positively charged cells may be added to a solution of the negatively charged cells. Or, for example, a solution of the negatively charged cells may be added to a solution of the positively charged cells. Or, for example, solutions of the positively charged cells and the negatively charged cells may be added simultaneously to a receptacle. The core of the aggregate is formed by the cell type which is present in the lower amount. Suitable ratios of core to coating cell type are 1:8 to 1.4:100.

For example, the negatively charged cells may be placed in a 5 ml microcentrifuge tube. The mixer may be mounted such that the mixing head is placed at the bottom of the conical shaped part of the 5 ml microcentrifuge tube. The mixer may be set at, for example, 2000 to 8000 rpm, for example 3000 to 6000 rpm, suitably 5000 rpm. The positively charged cells can then be added using a syringe pump at, for example, 50 to 500 μl h⁻¹, for example 100 to 300 μl h⁻¹, for example about 200 μl h⁻¹. Depending on the amount to be added, such addition may continue for, for example, 5 to 60 minutes, for example 10 to 40 minutes, suitably about 20 min.

The mixing of these solutions forms a suspension of aggregated cells and solitary cells. The amount of aggregation can be controlled by adjustment of the concentration of each cell type, the flow rate of the cells that are added to the suspension of oppositely charged cells, mixing/stirring speed (to ensure a well distributed suspension of cells), amount of added oppositely charged cells to suspension of cell, and ionic strength of cell surface. This allows for control of the number of aggregates in the solution.

Aggregation can be monitored using subsamples by, for example, light microscopy, dynamic light scattering size distribution or flow cytometry procedures. Light microscopy involves finding aggregates at lower magnifications and then determining the distribution of cells within the aggregates. The cells, suitably the cells forming the core of the aggregates, can be fluorescently labelled prior to aggregation, and distribution can be monitored by fluorescent microscopy. Dynamic light scattering size distribution exploits the thermal random movement wherein larger particles move further in one direction due to gravitation force. Therefore, the relative number of aggregates of a determined size can be estimated. In flow cytometry procedures one of the cells must be labelled fluorescently. Cells and aggregates of cells pass through a beam and scatter light. The side and forward scatter can show the size of the aggregate and the fluorescent signal determines at which size the fluorescent cores are formed. Accordingly, number and size of aggregates, and number of non-aggregated cells can be determined.

### Layering

The aggregates themselves comprise a core or centre of a cell or small flocculation of cells having one surface potential (positive, by surface modification, or negative, naturally or by surface modification), with a layer or coating of the oppositely charged/opposite surface potential cells (negative or positive) upon it.

That layer or coating may itself have a further layer or coating of cells upon it, having the opposite surface potential (positive or negative).

This layering of cells, and accordingly the size of the eventual product aggregates, can be controlled by using sequential alternating cell coating steps.

For example, a series involving coating natural/uncoated cells with positively charged cells; followed by coating with negatively charged cells; followed by coating with positively charged cells leads to aggregates which have a negative core and then three layers on top. In this way the species of cell that composes each layer can be controlled, enabling a combination of different cell layers to form multistep biocatalytic aggregates. The greater the number of layers, the more biotechnological processes can be combined in a very small space to work simultaneously, without the need for separated reactors or conditions. A suitable number of layers is 2 to 20.

In the above discussions, it is explained how the surface potential of a cell can be switched, from negative to positive, and indeed switched back to negative by addition of a further polyelectrolyte layer.

It is envisaged that repeated addition of layers of polyelectrolytes, to form layers of alternating charge on a single cell, by the procedures set out above, may be possible. This can control the individual size of the surface modified cells, which thus affects the thickness or size of the layer(s) in the aggregate formed using those cells.

By suitable layering of polyelectrolytes on cells, followed by suitable layering of the cells to form aggregates, the present techniques allow for great control of the positioning of cells within an aggregate, their concentration therewithin, the size of those aggregates and the thicknesses of the cell layers.

### Separating Aggregates

Once aggregates have been formed, for example within a solution, they can be removed or separated from it.

Suitable techniques include size exclusion, differential centrifugation in gradients, filtration, dielectrophoretic separation or dielectric separation due to the different charge distribution in aggregates than in individual cells and bulk density properties of aggregates. Cells can be also distinguished from aggregates via flow cytometry, and so the aggregates can be separated by flow sorters.

To increase the ease of separability, and the yield of such a procedure, the inventors have found that it is advantageous to make the aggregates themselves magnetic. This permits separation using a magnet to draw the aggregates to it, allowing for easier removal of the rest of the solution (for example) and more effective washing of the remainder (because the aggregates are magnetically held).

This can be achieved by, for example, fully or partially coating certain cells with a magnetic material. For example, the positively charged cells, or the negatively charged cells, may be coated with particles of a magnetic material. In some embodiments the positively charged particles are coated fully or partially with the magnetic material.

Magnetic particles are well known. Suitable particles include, for example, paramagnetic particles such as Fe₃O₄ particles.

The particles may be nanoparticles, for example, having a particle diameter of 5 nm or more and 1000 nm or less. The particle diameter may be measured by dynamic light scattering or electron microscopy.

Such a layer can be added by mixing a solution of the magnetic particles with a solution of the cells to be coated.

For example, to cells coated with a positive polyelectrolyte, a suspension containing Fe₃O₄ can be added. The suspension may contain, for example, 0.25% magnetite. This is added in an amount of around 1:1 v/v. The Fe₃O₄ particles then coat onto the positively charged cells.

To separate out cells with a negative surface potential, the magnetites can be pre-treated to change the surface charge to a positive one and used in the same way as above.

A permanent magnetic field can then be used to hold the coated cells while the uncoated cells are washed out.

After an initial wash, the remainder (magnetic cells plus unattached Fe₃O₄) can be suspended in solution, for example a 0.9% NaCl solution in water. This can then be centrifuged (at, for example, 1000-2000 g for 2-3 minutes) to remove the unattached Fe₃O₄.

The thus prepared cells, with an Fe₃O₄ coating, can be recoated by the procedures set out above for changing or enhancing the charge of cells, to give the desired surface charge/potential.

For example, the prepared cells can be suspended in 0.9% NaCl solution in water and a solution of polyelectrolyte added to them. Where the cells were positively charged before the magnetic coating was added, a positive polyelectrolyte can be used to re-impart a positive surface charge/potential.

After that, the magnetic cells can be isolated by removal of the unused polyelectrolyte by centrifugation and washing (for example 3-4 times, using 0.9% NaCl solution in water).

The magnetic cells can then be used for formation of aggregates, or indeed for addition of further polyelectrolyte layers, as described herein.

When aggregates are made using such magnetic cells, the separation of the aggregates is simplified and improved. The suspension, comprising aggregates as well as solitary coating cells, which are present in excess, can be exposed to a permanent magnet (for example, 1T). The solution of unattached aggregates can then be removed. The unattached cells and aggregates can gradually be washed out, with the aggregates being kept ‘wet’ (or at least not dry) by addition of NaCl solution in deionised water at a maximum of 1.5% NaCl.

Two or more such washes can remove the solitary cells remaining.

The permanent magnet can then be removed, and the aggregates resuspended, for example in NaCl solution at maximum 1.2% NaCl in deionised water. The aggregates are then ready for further addition of cell layers as described herein, if wanted.

The prepared aggregates can be then physically stabilised by encapsulation either using additional polyelectrolyte coating over the aggregates, covalent crosslinking or using matrix stabilization such as alginate, polyacrylamide beads or other gels. Mechanical encapsulation enables the aggregates to maintain stability when cells start to divide.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the present disclosure in diverse forms thereof.

While the present disclosure has been described in conjunction with the exemplary embodiments described above, many equivalent modifications and variations will be apparent to those skilled in the art when given this disclosure. Accordingly, the exemplary embodiments of the present disclosure set forth above are considered to be illustrative and not limiting. Various changes to the described embodiments may be made without departing from the spirit and scope of the present disclosure.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### EXAMPLES

### Example 1

### (i) Preparation of Cells

The goal of this procedure is to define appropriate growth stage of the cell to achieve the lowest electrophoretic softness (λ⁻¹) as well as the highest surface charge (ZN).

Nonmotile cells of Escherichia coli top 10 strain (F-mcrA Δ(mrr-hsdRMS-mcrBC) ϕ80lacZΔM15 ΔlacX74 recA1 araD139 Δ(ara leu) 7697 galU galK rpsL (StrR) endA1 nupG) were transformed with pRSET-emGFP plasmid (Thermo Fisher Scientific Corp.) using standard electroporation procedures. The plasmid contained T7 promoter regions upstream of the emGFP reporter gene and ApR cassette. Since cells are deficient in T7 polymerase, the GFP is transcribed due only to promotor leakage. The transformants were cultivated at 37° C. on nutrient agar (NA) plates (Sigma-Aldrich) supplemented with ampicillin (100 μg/ml, Sigma-Aldrich)—NAamp.

For efficient polyelectrolyte deposition, the electrostatic properties of the surface of bacterial cell in different growth stages were determined. The charge densities (ZN) and the electrostatic softness parameter (λ⁻¹) of bacterial cells were determined from a nonlinear regression analysis of ionic strength dependant electrophoretic mobilities (ISDEM) of bacterial cells by using Ohshima's soft particle equation as a model.

To obtain the ISDEM, the cultures were washed 3 times by centrifugation (3000 g for 3 min), pouring out of the supernatant, and resuspension in 0.00062M NaCl solution prepared from deionised water (18 MOhm), being careful not to disturb the pellet. Then 100 μl of washed culture was mixed with 900 μl of 0.00062 M NaCl solution and the cells were properly dispersed using maximum vortexing of the suspension of cells for 10 s. The electrophoretic mobilities of bacterial cells were measured using an ELS device (Zetasizer Nano, Malvern, USA). The ionic strength of the suspension of bacterial cells within the measuring cuvette was automatically altered with titration using an MPT-2 titrator. Measurements were made within the linear gradient of ionic strengths of NaCl from 0.00062 M to 0.11 M in 12 steps of 0.0091 M per step by addition of the 0.155 M solution of NaCl. The data was obtained from 3 experimental replicates where each of the measurements was acquired from the accumulated values of 70 separated ELS values. In all ELS measurements the polarities of electrodes were fixed, with an automatically adjustable voltage. The approximate values of ionic strengths were obtained from the titrator and the exact values for the cell suspensions within cuvettes were determined on a basis of conductivity values obtained in each ELS measurement. The ionic strengths in the ELS measurements were calculated on a basis of the standard curve values obtained from the measurement of the conductivities of different concentrations of NaCl solutions in water.

When the appropriate growth stage is determined the bacterial cells must be prepared from the fresh culture following the protocol: from the overnight liquid cultures obtained from a single colony grown on solid medium 1 mL of this culture is transferred into the 100 mL fresh medium and incubated with shaking at 37° C. and 150 rpm until reaching the optical densities corresponding to the previously determined appropriate growth stage.

E. coli cells were grown at 37° C. by shaking at 150 rpm until OD660=0.2 was reached. Cells were concentrated by centrifugation of 50 mL of the culture at 5000 g for 6 min. To wash out residues from the medium the pellet was washed three times by resuspension in 30 mL of 0.9% NaCl solution and centrifugation of the suspension at 3000 g for 3 min.

### (ii) Modification of Surface Potential of Cells

In this example E. coli cells are used.

To make the surface potential of the cells positive, poly(ethyleneimine) PEs (PEI, MW=750,000 g/mol) was used. To make the surface potential of the cell negative, charged sodium poly(styrene sulfonate) (PSS, MW=70,000 g/mol) was used.

The solutions of PEs in Milli-Q (ultrapure water type 1) water (2.5 mg/ml, pH 7 adjusted by NaOH or HCl) were prepared by solubilizing PEs, initially by stirring and then by the sonication (35 kHz, 100 W) for 15 min. In experiments using labeled PEI with tetramethylrhodamine isothiocyanate (TRITC), the PEI was labeled using NHS ester labeling of amino biomolecules. Briefly, the TRITC (11 mg in 11 mL DMSO solution) was added to a PEI solution (2.5 mg/ml in 20 mL of water) in a 50 mL tube and stirred at room temperature for 4 h. To remove the residual dye from the solution, after labeling the solution was dialyzed for 3 days using a dialysis tube (Orange Scientific) with nominal molecular weight limits between 12 and 14 kDa.

Before the deposition of the polyelectrolyte on the surface of cells, cells were exposed to the ultrasound at 80 kHz, 100% power, for 3 minutes. The polyelectrolyte was deposited on a layer by layer basis.

To prevent formation of aggregates of the cells, the parameters for efficient single cell PE deposition was determined. Polyethyleneimine (PEI) was deposited on the cells by adding a 0.25% solution of PEI at pH 7 (adjusted with HCl) in Milli-Q water to the washed cells (OD660 1.2) in a 1:1 v/v ratio. This suspension was stirred at room temperature for 5 min. Unattached PEI was removed from the suspension by centrifugation at 900 g for 2 min. The obtained pellet was washed twice by gentle addition of 1 ml of 0.9% NaCl over the pellet taking care to avoid disturbing it. After washing, the PEI covered cells were resuspended in 0.9% NaCl solution.

A PSS layer was deposited on the PEI covered suspension using a 0.25% w/v solution in Milli-Q water of PSS at pH 7 (adjusted with NaOH) in a 1:1 v/v ratio using the same procedure as above, except that 3 min centrifugation at 1500 g was used to obtain a sufficiently firm pellet to permit washing by pipetting.

By repetition of these two steps, four such layers were deposited on the surface of bacterial cells while keeping their aggregation, as monitored by microscopy, low.

Such prepared cells were then exposed to an ultrasound waterbath at 80 kHz, using 100% power, for 3 minutes.

(iii) Aggregation of Cells

The negatively charged cells with four layers of polyelectrolyte, prepared as above, were put into a 5 ml microcentrifuge tube. The mixer was mounted such that the mixing head was placed at the narrow end of the conical shaped part of the 5 ml microcentrifuge tube. The mixer was set at 5000 rpm. The positively charged E. coli cells with one polyelectrolyte layer, prepared as described in ii) above, were slowly added using a syringe pump at 200 μl h⁻¹ for 20 min. A suspension of aggregated cells and solitary cells was formed.

### (iv) Extraction of Aggregates

For extraction of aggregates using a magnet, E. coli cells with one positive polyelectrolyte layer were prepared as above and modified prior to aggregation. A suspension of 10-20 nm large citrate stabilized magnetite particles were added to the E. coli cells. Over that layer a negative layer was deposited and then a positive layer was deposited using the same procedure as ii) above. Aggregation was carried out as described in iii).

The suspension comprising aggregated as well as solitary cells was exposed to a strong permanent magnet (1 T), and left for 10 minutes under such exposure. This separated the magnetic aggregated cells. The solution of unattached aggregates was then taken out.

To wash out the remaining solitary cells from the separated aggregates, washing with 1.5% NaCl solution in water was conducted three times.

The tube was then removed from the permanent magnet and the remaining separated aggregates were resuspended in 100 μl NaCl solution at 1.2% NaCl in deionised water.

### REFERENCES

A number of publications are cited above in order to more fully describe and disclose the aspects of the disclosed embodiments and the state of the art to which the present disclosure pertains. The entirety of each of these references is incorporated herein.

1. A method for making an aggregation of cells, the method comprising the steps of:
(a) treating a first portion of cells with a positively charged polyelectrolyte to form a first portion of surface modified cells; and
(b) mixing the first portion of surface modified cells with a second portion of cells, to form aggregations of cells each having a negative surface potential and each comprising a layer of cells on the surface of a surface modified cell.

2. The method according to claim 1, wherein the second portion of cells is not surface modified.

3. The method according to claim 1, wherein the second portion of cells comprises a second portion of surface modified cells formed by a method comprising the steps of:
(i) treating cells with a positively charged polyelectrolyte; and then
(ii) treating said cells with a negatively charged polyelectrolyte, to form the second portion of surface modified cells.

4. The method according to claim 1, the first portion of surface modified cells or the second portion of cells is treated with magnetic particles to coat their surface with such particles or embed such particles onto their surface, before the step of mixing the first portion of surface modified cells with the second portion of cells.

5. The method according to claim 4, wherein the magnetic particles are paramagnetic.

6. The method according to claim 5, wherein the magnetic particles are Fe3O4 particles.

7. The method according to claim 1, wherein each positively charged polyelectrolyte is individually selected from polyethyleneimine, polydiallyldimethylammonium chloride, poly(allylamine hydrochloride), chitosan, cationic starch and amino cellulose.

8. The method according to claim 1, wherein each negatively charged polyelectrolyte is individually selected from polystyrene sulfonate, polyacrylic acid, alginate or cellulose.

9. The method according to claim 1, wherein the first portion of cells and the second portion of cells are of different types.

10. The method according to claim 1, wherein each cell is a microbial cell, preferably a bacterial cell.

11. A method for making an aggregation of cells, the method comprising:
treating an aggregation of cells with a negative surface potential, as made by the method of claim 1, with a portion of cells having a positive surface potential, to form an aggregation of cells each having a positive surface potential and each comprising a layer of cells having a positive surface potential, on the surface of a layer of the second portion of cells, on the surface of a surface modified cell.
